(19) United States
(12) Patent Application Publication
(10) Pub. No.: US 2019/0160198 A1
(43) Pub. Date: May 30, 2019

(54) BIOABSORBABLE DERMAL REGENERATION MATRIX AND METHODS OF MAKING AND USING SAME

(71) Applicant: Duke University, Durham, NC (US)

(72) Inventors: Howard Levinson, Durham, NC (US); Elizabeth R. Lorden, Durham, NC (US); David A. Brown, Durham, NC (US)

(73) Assignee: Duke University, Durham, NC (US)

(21) Appl. No.: 16/318,855

(22) PCT Filed: Jul. 18, 2017
(86) PCT No.: PCT/US2017/042550

§ 371 Date: Jan. 18, 2019

Related U.S. Application Data
(60) Provisional application No. 62/363,403, filed on Jul. 18, 2016.

(51) Int. Cl. A61L 15/32; A61L 27/58; A61L 27/56; A61L 27/54; A61L 27/34; A61L 27/18; A61L 26/00

(52) U.S. Cl. CPC A61L 15/325; A61L 27/58; A61L 27/56; A61L 27/54; C08L 89/06; A61L 27/18; A61L 26/00; A61L 2430/34; A61L 27/34

(57) ABSTRACT

Disclosed herein are wound healing products and methods of making and using the same. The wound healing product comprises a porous scaffold and collagen bound thereon. The scaffold may comprise a poly(L-lactide-co-ε-caprolactone) polymer (PLCL) substrate. In some embodiments, the PLCL substrate comprises a mixture of poly(lactic acid) (PLA) and poly(ε-caprolactone) (PLC) and wherein the PLA and PLC are present in a ratio of about 60:40 to about 40:60. In some embodiments, the collagen is collagen I or collagen III. The scaffold may also have a thickness of at least 0.2 mm.

### CROSS REFERENCE TO RELATED APPLICATIONS

This application claims benefit of priority to U.S. Provisional Application Ser. No. 62/363,403 filed 18 Jul. 2016, the contents of which are incorporated by reference in its entirety.

### FIELD OF INVENTION

The technology generally relates to bioabsorbable dermal regeneration matrices and methods of making and using the same. More specifically the technology relates to wound healing products comprising an absorbable polymeric biomaterial fabricated into a porous scaffold coated with collagen.

### BACKGROUND

Scarring is a significant medical problem that affects more than 80 million people worldwide annually and can have many etiologies. Currently available skin substitutes for wound healing have suboptimal degradation rates leading to the formation of hypertrophic scars that limit mobility, impact quality of life and cost millions of dollars per year in surgical treatment and physical therapy. These limitations include low elasticity and strength, poor incorporation into surrounding tissue, and rapid degradation time. The undesirable mechanical properties of existing products relegate their use to relatively static areas of wound healing and away from dynamic areas such as joints surfaces. In the case that existing products are used in these areas, the joints or muscles must be immobilized for some period of time, which can result in substantial functional morbidity including a permanent loss in range of motion. Thus, there is a need for wound healing products having superior mechanical properties and that promote substantive biologic incorporation into surrounding tissues.

### SUMMARY OF THE INVENTION

Disclosed herein are wound healing products and methods of making and using the same. The wound healing product comprises a porous scaffold and collagen bound thereon. The scaffold may comprise a poly(L-lactide-co-ε-caprolactone) polymer (PLCL) substrate. In some embodiments, the PLCL substrate comprises a mixture of poly(lactic acid) (PLA) and poly(ε-caprolactone) (PLC) and wherein the PLA and PLC are present in a ratio of about 60:40 to about 40:60. In some embodiments, the collagen is collagen I or collagen III. The scaffold may also have a thickness of at least 0.2 mm. In some embodiments, the scaffold has a thickness of at least 1.0 mm. In some embodiments, the scaffold further comprises a porogen and/or sucrose.

The porous scaffold may have a porosity of between 60% and about 95%. In some embodiments, the scaffold has a porosity of between about 80% and about 95%. The porous scaffold may have a pore interconnectivity of greater than about 80%. In some embodiments, the scaffold has a pore interconnectivity of greater than about 90%. The porous scaffold may also have a mean pore diameter of between about 50 microns and about 250 microns.

Another aspect of the invention is a method for preparing a porous scaffold or a wound healing product. The method comprises evaporating a polymer slurry solvent from a polymer slurry within a mold to prepare a polymer substrate surrounding a porogen, wherein the polymer slurry comprises the polymer slurry solvent, the porogen insoluble in the polymer slurry solvent, a solvent-soluble polymer, and a surfactant; and dissolving the porogen with a porogen solvent to prepare a polymer substrate having a plurality of pores. The method may further comprises etching the polymer substrate having a plurality of pores with an oxygen plasma to prepare an etched polymer substrate; and reacting collagen with the etched polymer substrate to prepare the wound healing product.

The polymer slurry may be prepared by mixing the solvent-soluble polymer and the polymer slurry solvent in a percent weight/weight of the solvent-soluble polymer to the polymer slurry solvent between about 10.0% to about 20.0%. The polymer slurry may be prepared by mixing the polymer and porogen in a percent weight/weight of the solvent-soluble polymer to the porogen of about 5.0% to about 70.0%. The polymer slurry may prepared by mixing the porogen and the surfactant in a percent weight/weight of the surfactant to the porogen of about 10.0% to about 20.0%.

The porogen may comprise a water-soluble sugar or salt. In some embodiments, the porogen comprises sucrose. The pogogen may be selected by size exclusion. For example the porogen may be selected by size-exclusion sieving.

In some embodiments, the reacting step comprises contacting the etched polymer substrate with 1-ethyl-3-(3-dimethylaminopropyl)carbodiimide hydrochloride (EDC) to prepare an O-acylisurea intermediate and displacing the O-acylisurea intermediate with a primary amine of the collagen and/or stabilizing an O-acylisurea intermediate with N-hydroxysuccinimide (NHS).

In some embodiments, the etching step comprises contacting the porous scaffold with an oxygen plasma having an sufficient power for a sufficient time to oxidize the surface of the porous scaffold.

In some embodiments, the method may further comprise sterilizing the porous scaffold or wound healing product. The sterilizing step may comprise contacting the porous scaffold or the wound healing product with a sterilization fluid for a time sufficient to sterilize the porous scaffold or wound healing product.

Another aspect of the invention is a method of promoting wound healing in a subject. The method comprises providing any of the porous scaffolds or a wound healing products described above where the porous scaffold or wound healing product is configured to be implanted at the site of a wound or beneath an applied skin graft of the subject. In some embodiments, the product is configured to be positioned between the muscle and the subcutaneous tissue of the subject.

The wound may be a chronic wound, a surgical wound, or fibrosis. Examples of chronic wounds include venous stasis ulcers and diabetic foot ulcers. Examples of surgical wounds include wounds resulting from aesthetic surgery, hernia repair, dura repair, orbital floor repair, breast reconstruction, urological repair, gynecological repair, or any combination thereof. Examples of fibrosis include fibrosis resulting from trauma, thermal injury, or radiation.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates an exemplary embodiment of a SCPL process.

FIG. 2 illustrates an exemplary use of the wound healing products described herein.

FIG. 3A-3B shows SEM images of porous PLCL scaffolds with 200× magnification (FIG. 3A) and 1000× magnification (FIG. 3B).

FIG. 4 shows elastic modulus measurements of solvents casted-particulate leached (SCPL) PLCL scaffolds versus other biomaterials standards.

FIG. 5 shows ultimate tensile strength measurements of solvents casted-particulate leached (SCPL) PLCL scaffolds versus other biomaterials standards.

FIG. 6 shows elongation at break measurements of solvents casted-particulate leached (SCPL) PLCL scaffolds versus other biomaterials standards.

FIG. 7 shows representative z-stack images of collagen in the center plane of a PLCL scaffold.

FIGS. 8A-8B shows PLCL scaffolds implanted in a murine HSc model for 2 months demonstrate anti-contraction effects at 20× and 40× magnification, respectively.

FIGS. 9A-9D shows explanted scaffolds from a mouse after 2 weeks stained with H&E for a 70% porosity PLCL scaffold, a 80% porosity PLCL scaffold, a 90% porosity PLCL scaffold, and an Integra™ scaffold, respectively.

FIGS. 10A-10D shows explanted scaffolds from a mouse after 4 weeks stained with H&E for a 70% porosity PLCL scaffold, a 80% porosity PLCL scaffold, a 90% porosity PLCL scaffold, and an Integra™ scaffold, respectively.

### DETAILED DESCRIPTION OF THE INVENTION

Bioabsorbable dermal regeneration matrices and methods of making and using the same are disclosed herein. The matrices are wound healing products that promote skin regeneration and minimize scarring. To achieve this goal, the development of scaffolds comprising a viscoelastic copolymer is described herein. As shown in the Examples that follow, poly(l-lactide-co-ε-caprolactone) (PLCL) scaffolds described herein contains favorable physicomechanical properties akin to unwounded skin, promote tissue in-growth that is important for sustaining a skin graft, lasts 6-12 months in vivo throughout wound healing, and PLCL prevents hypertrophic scar contracture (HSc) in vivo.

Dermal scarring affects more than 80 million people worldwide annually. In severe burns, more than 40% of patients develop hypertrophic scar contraction, which leads to hypertrophic scar contractures (HSc). The term “hypertrophic scar contraction” refers to the process of a hypertrophic scar reducing in size. The term “hypertrophic scar contracture” or “HSc” refers to the shrunken hypertrophic scar. The area of the scar will be significantly less than the area of the initial injury causing tightness in surrounding skin and webbing across joints. HSc are stiff, shrunken scars that limit mobility, impact quality of life and cost millions of dollars per year in surgical treatment and physical therapy. HSc are caused by increased mechanical tension and occur 6-12 months after injury.

HSc occurs during ‘adult healing’ in the 6-12 months post-wounding, as a result of persistent, incremental scar contraction. Scar contraction occurs because large wounds are amorphous and lack the physicomechanical cues indicative of scarless ‘fetal’ regeneration. Without physicomechanical cues for regeneration, adult healing ensues. Whereas attenuated mechanical tension promotes fetal repair and restoration of normal skin anatomy, in the adult healing response, fibroblasts populate the wound and deposit extracellular matrix (ECM) elements which do not shield the cells from stress until sufficient amounts of collagen are deposited and cross-linked over 3-6 months. “Extracellular matrix” or “ECM” refers to a collection of proteins and molecules secreted by cells which provide the structural and biochemical support to all tissues within the body. In the skin, the ECM is supported by elastin and glycosaminoglycans, but is primarily made up of collagen.

Mechanical tension stimulates inflammation and the differentiation of fibroblasts into myofibroblasts (identified by alpha-smooth muscle actin (αSMA) expression). “Fibroblast” refers to the most predominant cell type of the skin and connective tissue which is capable of synthesizing the extracellular matrix and collagen and “myofibroblast” refers to a cell that is in between a fibroblast and a smooth muscle cell in differentiation. Myofibroblasts are a more contractile version of the fibroblast and are not typically found in the skin. Fibroblasts turn into myofibroblasts when they encounter mechanical tension (pulling) or wound-related cytokines (chemicals). Myofibroblasts play a critical role in wound healing and HSc by serving to physically contract the wound, secrete new ECM, and remodel the ECM.

“Alpha-smooth muscle actin” or “αSMA” refers to a protein that is characteristically expressed by myofibroblasts and plays a key role in their contractility. Myofibroblasts are present in contracting scars for 6 months post-wounding. After 6 months, the ECM is crosslinked and stabilized and cells are stress shielded from mechanical tension. Stress shielding signals myofibroblast apoptosis, indicating completion of adult wound healing.

Current tissue engineered scaffolds, such as Integra™, have mechanical properties akin to unwounded skin, but the collagen based scaffolds rapidly degrade over 2 months, being too short-lived to prevent HSc. The key to preventing HSc is having a biocompatible scaffold which degrades over many months.

Skin scaffolds are effective at promoting regeneration for the 4-8 weeks they persist in vivo. During this time they possess the physicomechanical cues for regeneration and prevention of myofibroblast formation. The 3D scaffold filament structure guides fibroblast and endothelial cell infiltration to recapitulate unwounded skin. The filaments act like mechanical rebar, which provide elasticity to the deposited ECM; akin to how rebar enhance mechanical performance of cement buildings and roads by increasing elasticity. A comparative study of 5 dermal substitutes (Integra™, ProDerm™, Renoskin™, Matriderm™, and Hyalomatrix™ PA) demonstrated that stress shielding benefits for all of the substitutes were lost upon degradation at 2 months after application (Philandrianos C et al. Comparison of five dermal substitutes in full-thickness skin wound healing in a porcine model. Burns 2012; 38:820-9). This degradation occurs prior to ECM stabilization, and mechanical tension returns and adult scarring ensues in the following 6-12 months.

It had been demonstrated in International Patent Publication No. WO 2016/077480 to Levinson et al. (the contents of which are incorporated by reference in its entirety) that electrospun ˜100 μm thick PLCL scaffolds possess appropriate mechanical properties for implantation beneath skin grafts and inhibition of HSc in mice. However, electrospinning cannot be used to generate scaffolds at a thickness necessary for large animal studies due to the great voltage gradient required for scaffold production. To overcome this hurdle a solvent casting and particulate leaching (SCPL) is described herein to create 3D porous PLCL scaffolds. Briefly, this method involves the mixing of polymers with particles within a solvent followed by casting into a mold. Water is then passed through the mold to leach out the particles, resulting in an interconnected porous 3D scaffold. As shown in the Examples below, the SCPL method has successfully been used to create PLCL scaffolds several millimeters thick, which have appropriate mechanical properties and tunable porosity for the preparation of wound healing products.

### Wound Healing Products

“Wound healing products” of the present invention comprise a porous scaffold and a coating thereon. Wound healing products are capable of promoting wound healing, and may be configured to be implanted at the site of a wound or beneath an applied skin graft of a subject.

“Scaffolds” are materials and/or compositions that provide structural support for cells in the body of a subject. “Subject” refers to both human and nonhuman animals. The term “non-human animals” of the disclosure includes all vertebrates, e.g., mammals and non-mammals, such as nonhuman primates, sheep, dog, cat, horse, cow, chickens, amphibians, reptiles, and the like. In some embodiments, the subject is a human. In certain embodiments, the subject is a human who has a wound.

Scaffolds of the present invention may comprise a polymer. “Polymer” refers to a compound having many repeating units. As used herein, polymers can be synthetic (man-made), or biologic (derived from animals, or recombinantly produced). Synthetic polymers are low cost, have a long shelf life, and possess tunable degradation rates and mechanical properties. However, synthetic polymers do not have cell-specific recognition sites. As a result, certain embodiments of the disclosure provide the biocompatible scaffold that is coated with one or more extracellular matrix proteins. Biologic polymers are high cost, have short shelf-life, possess possibilities of immune-reaction, have weak mechanical properties, and low tunability of degradation rate. However, they have cell-specific recognition sites which allow the body to interact with them readily and easily. The ECM component collagen can be considered a biologic polymer.

In some embodiments, such as those described in the Examples, the scaffold may comprise poly(l-lactide-co-ε-caprolactone) (PLCL), which comprises a mixture of poly(lactic acid) (PLA) and poly(ε-caprolactone) (PLC). PLCL is a biodegradable elastomer that has elasticity and mechanical strength similar to unwounded human skin. It is also biocompatible with tissue ingrowth, supports skin graft survival and has an extended degradation rate (6-12 months in vivo). This copolymer's elastomeric characteristics are due to phase separation of the crystalline PLA and the amorphous PCL segments, creating hard and soft domains akin to those observed in elastomeric polyurethanes. It degrades into acidic breakdown products of lactic acid and caprylic acid, which are then secreted from the body as hydroxy acids or metabolized into CO₂ and water. PLCL is not known to illicit a strong immune response.

The mixture of PLA and PLC in PLCL may be present in a ratio of about 5:95 to about 95:5 by weight. In some embodiments, the PLCL comprises a mixture of PLA and PLC in a ratio of about 10:90 to about 90:10, or about 20:80 to about 80:20, or about 25:75 to about 75:25, or about 30:70 to about 70:30, or about 40:60 to about 60:40, or about 42:58 to about 58:42, or about 45:55 to about 55:45, or about 47:53 to about 53:47, or about 48:52 to about 52:48, or about 49:51 to about 51:49 by weight. In a particular embodiment, the PLCL comprises a 50:50 mixture of PLA and PLC. It will be appreciated that altering the ratios of PLA and PLC will provide the scaffold with different properties, such as elasticity, degradation rate, toughness and the like. Some of these properties may be beneficial depending on the use, and will be readily appreciated by one skilled in the art. Hence, PLCL scaffolds having varying ratios of PLA and PLC are contemplated and intended to be within the scope of the present disclosure.

The polymer may have any molecular weight suitable for preparing the scaffold. In some embodiments, the polymer has a mass average molar mass (M_{w}) of at least 100 kDa. In particular embodiments, the polymer has a M_{w} of at least about 150 kDa, at least about 200 kDA, at least about 250 kDa, at least about 300 kDa, at least about 350 kDa, at least about 400 kDA, at least about 450 kDa, or at least about 500 kDa.

Scaffolds of the present invention may further comprise a coating to facilitate cellular attachment, proliferation, and differentiation. The surface chemistry may allow cells to recognize the scaffold through naturally occurring cell surface receptors such as integrins. Without a recognizable surface coating cells cannot interact with the scaffold and the scaffold will not bioincorporate. The coating may comprise a collagen or a carbohydrate polymer. “Collagen” refers to a protein synthesized by cells during wound healing and the primary component of the extracellular matrix of skin. In some embodiments, the scaffolds are coated with a collagen, e.g., collagen I or collagen III. In other embodiments, the scaffolds may be coated with a carbohydrate polymer, e.g., hyaluronic acid. The coating may be covalently bound to a substrate. The coating may comprise a moiety capable of reacting with the substrate to covalently bind the coating to the substrate. As shown in the Examples, a primary amine of collagen may be reacted with O-acylisurea intermediate to covalently bind collagen to an oxygen plasma treated PLCL scaffold.

Scaffolds of the present invention are porous, comprising a plurality of pores providing void spaces within the scaffold, allowing for the in-growth of surrounding tissues. The “porosity” of a scaffold is a measure of percentage of void space in the scaffold, i.e. Porosity=100%×void volume/(void volume+polymer volume). Scaffolds of the present invention may have a porosity greater than 60%. In some embodiments, the scaffolds have a porosity of between about 60% and about 95%, about 65% and about 95%, about 70% and about 95%, about 80% and about 95%, about 85% and about 95%, or about 90% and about 95%, including porosities of about 85%, 86%, 87%, 88%, 89%, 90%, 91%, 92%, 93%, 94%, or 95%.

The “pore size” of the scaffold is a measure of the mean pore diameter. The Scaffolds of the present invention may have a mean pore diameter of between about 25 microns and about 300 microns. In some embodiments, the scaffold has a mean pore diameter between about 40 microns to about 250 microns, or about 50 microns to about 200 microns.

The “interconnectivity” of the scaffold is a measure of the percentage of pores accessible to a bath of molecules surrounding the scaffold, i.e., interconnectivity=100%×accessible void volume/total void volume. The scaffolds of the present invention may have an interconnectivity of at least about 80%. In some embodiments, the scaffolds have an interconnectivity of at least about 90%, at least about 95%, at least about 96%, at least about 97%, at least about 98%, or at least about 99%.

The scaffold may be prepared into any desirable 3D shape capable of being formed with a mold, including substantially cylindrical or parallelepipedic shapes.

The “thickness” of the scaffold is a measure of the shortest gross dimension of the scaffold. By way of example, the thickness of a cylindrical scaffold having a larger diameter than height will be determined by the height, but the thickness of a cylindrical scaffold having the a larger height than diameter will be determined by the diameter. By way of another example, the thickness of a parallelpipedic scaffold will be determined by the shortest distance between any two parallel faces. Scaffolds of the present invention may have a thickness of at least about 0.2 mm. In some embodiments, the scaffolds have a thickness of at least about 0.2 mm to about 10.0 mm, at least 0.5 mm to about 5.0 mm, at least about 1.0 mm to about 3.0 mm.

The “elastic modulus” is a measure of the scaffold's resistance to being deformed elastically when a stress is applied. The elastic modulus is defined as the slope of its stress-strain curve in the elastic deformation region. Scaffolds of the present invention may have an elastic modulus of between about 1.0 kPa to about 15.0 kPa. In some embodiments, the scaffold may have an elastic modulus of about 2.0±1.0 kPa, about 3.0±1.0 kPa, about 4.0±1.0 kPa, about 5.0±1.0 kPa, about 6.0±1.0 kPa, about 7.0±1.0 kPa, about 8.0±1.0 kPa, about 9.0±1.0 kPa, about 10.0±1.0 kPa, about 11.0±1.0 kPa, about 12.0±1.0 kPa, about 13.0±1.0 kPa, or about 14.0±1.0 kPa.

The “elongation at break,” also known as fracture strain, is the ratio between changed length and initial length after breakage of the test specimen. It expresses the capability of a material to resist changes of shape without crack formation. Scaffolds of the present invention may have an elongation at break of between about 400% to about 600% kPa. In some embodiments, the scaffolds may have an elongation of at break of between about 450% to about 550%.

The “ultimate tensile strength” is measured by the maximum stress that a material can withstand while being stretched or pulled before breaking. The scaffolds of the present invention may have an ultimate tensile strength of between about 100 kPa to 1200 kPa. In some embodiments, the scaffold may have an ultimate tensile strength of about 200±100 kPa, about 300±100 kPa, about 400±100 kPa, about 500±100 kPa, about 600±100 kPa, about 700±100 kPa, about 800±100 kPa, about 900±100 kPa, about 1000±100 kPa, or about 1100±100 kPa.

The scaffolds may further comprise a porogen used to prepare the porous scaffold, as described below. The presence of the porogen may be a result an incomplete dissolving of the porogen and/or being completely surrounded by polymer such that the porogen solvent is incapable of contacting the porogen to dissolve it. The porogen may be any of the porogen materials described below, including sucrose.

The porous scaffolds may be combined with one or more additional compounds. The one or more compounds may be coated onto the scaffold itself, or administered before, concurrently, and/or after the implanting of the scaffold within the wound. Further, the one or more compounds may be administered at the same time, sequentially, etc. The compounds may include any compound that promotes host/scaffold interactions and/or has anti-scarring and/or pro-wound healing properties. Examples include, but are not limited to, extracellular matrix proteins such as collagen and hyaluronic acid, anti-scarring compounds such as statins and losartan, pro-healing compounds such as peptides, nucleic acids, micro inhibitory RNAs (miRNAs), antibodies, growth factors and the like. One skilled in the art can readily determine the most optimal route of administration, dosage, and timing of administration of the said one or more additional compounds.

### Methods for Preparing Porous Scaffolds and Wound Healing Products

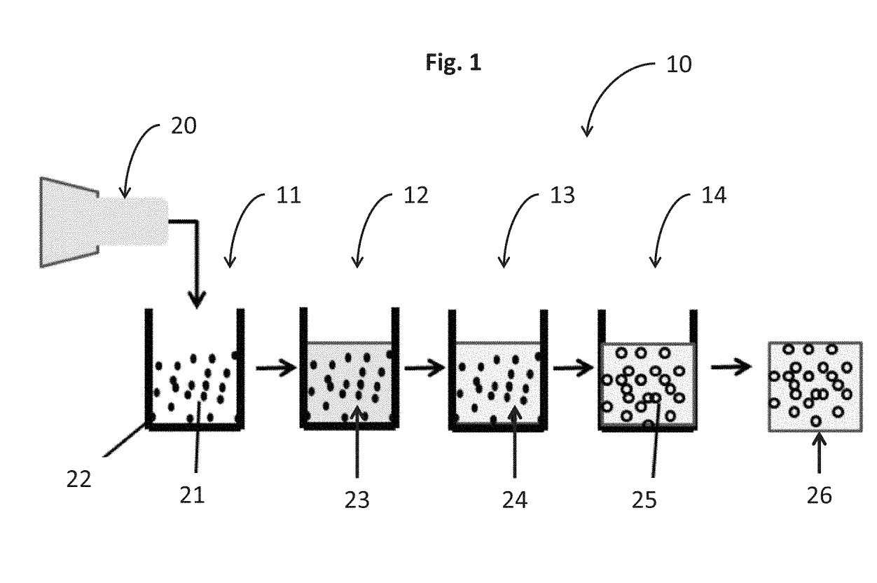

The wound healing products may be prepared by a solvent casting and particulate leaching (SCPL) process. “Solvent casting and particulate leaching” or “SCPL” is a process for making porous structures by first preparing a composite material consisting of a polymer and a porogen and then dissolving the porogen to leave behind the porous structure. The generalized process 10 is illustrated in FIG. 1. In a first step 11, a polymer solution 20 comprising a polymer slurry solvent and solvent-soluble polymer is mixed with a porogen 21 in a mold 22 to prepare a polymer slurry 23. In a second step 12 the solvent is allowed to evaporate from the polymer slurry 23 to prepare a composite material comprising a polymer substrate surrounding the porogen 24. In a third step 13, the composite material comprising a polymer substrate surrounding the porogen 24 is contacted with a porogen solvent capable of dissolving the porogen to prepare a polymer substrate having a plurality of pores 25. In a fourth step, the mold 22 may be removed to prepare a free polymer substrate 26.

The polymer slurry comprises a polymer solvent, a solvent-soluble polymer, and a porogen. In some embodiments, the polymer slurry further comprises a surfactant. Those of skill in the art will recognize that the polymer solvent, solvent-soluble polymer, porogen, and surfactant, if present, may be combined over wide ranges to affect the physicomechanical properties and/or degradation rates of the resulting scaffold.

The slurry may be prepared by mixing the solvent-soluble polymer and the polymer slurry solvent in a percent weight/weight of the solvent-soluble polymer to the polymer slurry solvent between about 10.0% to about 20.0%, including from about 12.0% to about 18.0%, or about 14.0% to about 16.0%. In some embodiments, the percent weight of the solvent-soluble polymer to the polymer slurry solvent is about 11.0%±1.0%, 12.0%±1.0%, 13.0%±1.0%, 14.0%±1.0%, 15.0%±1.0%, 16.0%±1.0%, 17.0%±1.0%, 18.0%±1.0%, or 19.0%±1.0%.

The polymer slurry may be prepared by mixing the polymer and porogen in a percent weight/weight of the solvent-soluble polymer to the porogen is between about 5.0% to about 70%. In some embodiments, the percent weight/weight of the solvent-soluble polymer to the porogen is between about 5.0% to about 60.0%, about 5.0% to about 50.0%, about 5.0% to about 40.0%, about 5.0% to about 35.0%, about 5.0% to about 30.0%, about 5.0% to about 25.0%, about 5.0% to about 20.0%, or about 5.0% to about 15.0%. The weight ratio of the polymer to the porogen may be used to control the porosity of the scaffold, and the porosity may be approximated as a function of those two variables.

If the surfactant is present, the polymer slurry is prepared by mixing the porogen and the surfactant in a percent weight/weight of the surfactant to the porogen between about 10.0% to about 20.0%, including from about 12.0% to about 18.0%, or about 14.0% to about 16.0%. In some embodiments, the percent weight of the surfactant to the porogen is about 11.0%±1.0%, 12.0%±1.0%, 13.0%±1.0%, 14.0%±1.0%, 15.0%±1.0%, 16.0%±1.0%, 17.0%±1.0%, 18.0%±1.0%, or 19.0%±1.0%.

“Solvent-soluble polymers” include any of the polymers described above, including PLCL polymers.

The “polymer solvent” many be any solvent capable of solvating the polymer to prepare a polymer solution When the polymer is PLCL, the polymer solvent is a solvent capable of solvating PLCL to prepare a PLCL solution. To facilitate evaporation, the polymer solvent should have a vapor pressure high enough to allow evaporation. In some embodiments, the polymer solvent is CHCl₃.

The polymer solvent may be evaporated from the polymer slurry by any method known in the art at atmospheric pressure or under vacuum for a sufficient length of time to allow the polymer to solidify or gel. In particular embodiments such as showing in the Examples, the polymer solvent is allowed to evaporate overnight in a fume hood.

The “porogen” is any material capable of being used the prepare pores in a polymer. Porogens may be gases, liquids, or a plurality of solid particles and should have low solubility in the polymer solvent so that a polymer slurry may be prepared. In some embodiments, the porogen comprises a water-soluble sugar or salt. In particular embodiments, the porogen may comprise a plurality of sucrose particles. In some embodiments, the porogen has a mean particle diameter of between about 25 microns to about 350 microns. In particular embodiments, the porogen has a mean particle diameter between about 50 microns and about 250 microns, about 50 microns and about 200 microns, about 125 microns to about 250 microns, or about 150 to about 250 microns.

When the porogen comprises a plurality of solid particles, the porogen may be selected to have a particular size. The porogen may be selected by size-exclusion, such as size-exclusion sieving. In some embodiments, one particle mesh may be used to exclude the largest or smallest particles by selecting the particles that do or do not pass through a particle mesh, respectively. If particles within a desired size range are desire, two particle meshes may be used to exclude both the largest and smallest particles. Particles meshes 45 to 500 may be used, including 45, 50, 60, 70, 80, 100, 120, 140, 170, 200, 230, 270, 325, 400, and 500. Pairings of these meshes may also be used, e.g., −45+500, −50+400, −60+325, −60+120, −70+270, or −70+100 mesh pairings.

The “surfactant” is a composition for lowering the surface tension at the interfaces between the components of the polymer slurry. The surfactant may improve the solubility of polymer, but should not dissolve the porogen. The surfactant may comprise methanol in some embodiments.

The “porogen solvent” is a composition capable of dissolving the porogen. Because it is undesirable for the porogen solvent to dissolve the polymer substrate after the polymer solvent has been evaporated, the porogen solvent should not appreciably dissolve the polymer substrate. In some embodiments, the porogen solvent comprises an aqueous solvent.

The method may further comprise a surface modification of the porous substrate to prepare a wound healing product. The surface modification may be the addition of a coating, such as collagen coatings described above, and/or modification of the surface of the porous substrate to allow for addition of a coating.

In some embodiments, surface modification may be accomplished with the use of an oxygen plasma treatment. The oxygen plasma may oxidize the surface of the porous scaffold. The oxygen plasma should have a power sufficient to oxidize the surface. For a porous scaffold comprising PLCL, the oxygen plasma will prepare carboxyl moieties on the surface of the porous scaffold. The oxygen plasma may have a power of between about 50 Watts to about 150 Watts, including between about 75 Watts and 100 Watts. The oxygen plasma may contact the porous surface for any time sufficient to oxidize the porous scaffold but less than a time where the oxygen plasma is substantially destructive to the porous surface. In some embodiments, the oxygen plasma is contacted with the porous surface for a time between about 30 seconds and about 90 seconds, including between about 45 seconds and about 60 seconds.

The surface of the porous scaffold may also be modified by 3-(ethyliminomethyleneamino)-N,N-dimethylpropan-1-amine hydrochloride (EDC). EDC may react with carboxyl moieties on the surface of the porous scaffold to prepare an O-acylisurea intermediate. O-acylisurea intermediates are reactive with primary amines. This allows for the covalent binding of the primary amine of a coating composition with the porous scaffold. This includes the covalent binding of a primary amine of collagen with the porous scaffold. N-hydroxysuccinimide (NHS) may also be used to stabilize an O-acylisurea intermediate to improve efficiency and prepare amine reactive intermediates.

The method may also include a sterilization step. In some embodiments, the porous scaffold or wound healing product is sterilized. The sterilization step may comprise contacting the wound healing product with a sterilization fluid for a time sufficient to sterilize the porous scaffold or wound healing product. The sterilization fluid may contact the porous scaffold or wound healing product for at least 15 minutes, but the sterilization fluid may contact the scaffold or product for longer periods such at least 20 minutes, at least 25 minutes, or at least 30 minutes. The sterilization fluid may comprise ethanol, including aqueous solutions of ethanol having sufficient concentrations of ethanol to sterilize the scaffold or product.

### Methods of Promoting Wound Healing

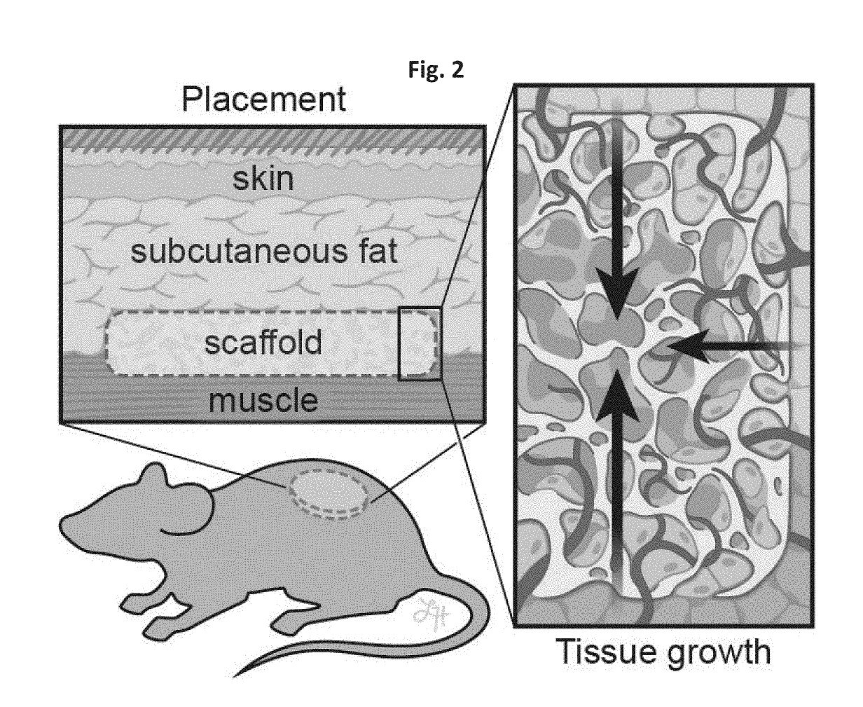

The wound healing products described herein may be used to promote wound healing in a subject. The wound healing products provided by the methods described herein may be configured to be implanted at the site of a wound or beneath an applied skin graft of the subject. In some embodiments, the wound healing product is configured to be positioned between the muscle and subcutaneous tissue of the subject. Without being bound to a particular theory, it is believed that implanting the wound healing products described herein promotes granulation and tissue formation and prevents skin graft contraction; minimizes mechanical strain transmission, and/or reduces inflammation and promotes ECM alignment thereby preventing and/or reducing scar contracture. An illustration of the use of the wound healing products described herein is illustrated in FIG. 2.

Any appropriate wound can be repaired according to the methods provided herein. For example, in some embodiments the wound may comprise a chronic wound, such as a venous stasis ulcer or a diabetic foot ulcer. In other embodiments, the wound comprises fibrosis following trauma, thermal injury or radiation damage. In yet other embodiments, the wound comprises a surgical wound, such as those following aesthetic surgery, hernia repair, dura repair, orbital floor repair, breast reconstruction, urological repair, gynecological repair, combinations thereof and the like.

In certain embodiments, the wound healing product is resorbed and/or remodeled by infiltrating components and supportive tissues that are generated or regenerated in accordance with the disclosed methods. To assist with the prevention of HSc, the wound healing products should retain favorable physicomechanical properties similar to unwounded skin for a period of at least 2 weeks in vivo. In some embodiments, the wound healing products retain favorable physicomechanical properties similar to unwounded skin for a period of at least 1 month, at least 2 months, at least 3 months, at least 4 months, at least 5 months, or at least 6 months in vivo. In some embodiments, less than 50% of the wound healing product is resorbed over a period of at least 1 month, at least 2 months, at least 3 months, at least 4 months, at least 5 months, or at least 6 months in vivo.

### Miscellaneous

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Fabrication of an Exemplary Wound Healing Product and its Characterization

### Scaffold Fabrication

Porous PLCL scaffolds were fabricated from 50/50 poly-L-lactide/poly-ε-caprolactone (generously provided by Youngmee Jung, Ph.D. of the Korea Institute of Science and Technology) by a solvents casting and particulate leaching process. PLCL (50% LA, 50% CL). Briefly, 1-lactide (100 mmol; Purac; Lincolnshire, Ill., USA) and ε-caprolactone (100 mmol; Sigma; St. Louis, Mo., USA) were polymerized at 150° C. for 24 h in the presence of stannous octoate (1 mmol, Sigma) as a catalyst. After being dissolved in chloroform, the polymer was precipitated in methanol, then dried under a vacuum for 72 h and stored in vacuum pack at −20° C. A 15% (w/w) polymer solution was then prepared by dissolving solid PLCL in chloroform. Subsequently, 0.5 g of the polymer solution was mixed with 1.48 g of sucrose (sieved to 150-250 microns) and 0.22 g methanol. The slurry was packed into cylindrical Teflon molds (10 mm diameter×2 mm thickness) and allowed to dry overnight in a fume hood. Scaffolds were then removed from the molds and leached in deionized water for 24 hours before analysis.

### Scanning Electron Microscopy

Fiber characteristics and scaffold thickness were analyzed using Scanning Electron Microscopy (FEI XL30 SEM-FEG) with the following settings: Voltage of 3 kV, working distance between 12-25 mm, and magnification between 200-5000×. The SE detector was used to best analyze changes in sample topography. Six separate samples were evaluated at three locations across each sample.

### Oxygen Plasma Etching

Samples were placed inside of a plasma etcher (Emitech K-1050×) and treated with reactive oxygen plasma for one minute at 75 watts. Following treatment, samples were immediately immersed in sterile water and subsequently sterilized in 70% ethanol for 20 minutes. Samples were rinsed thoroughly with water following sterilization.

### Collagen Coating

Covalent collagen coating was performed by EDC/NHS chemistry as previously described. This well-characterized method is biocompatible, non-cytotoxic, and does not include a linker-arm. Carbiodiamide is not incorporated into the covalent-linkage, allowing the collagen to directly coat scaffold. EDC reacts with carboxylic acid groups formed during reactive oxygen treatment to form an active O-acylisourea intermediate that is easily displaced by nucleophilic attack by primary amino groups. The O-acylisourea intermediate is unstable in aqueous solutions so the NHS (N-hydroxysuccinimide) is included in the reaction to stabilize the reactive group by forming intermediate succinimidyl ester groups. When collagen solution is introduced, a covalent reaction occurs between the amine groups of collagen and the active succinimidyl ester groups, resulting in formation of stable amide linkages on the polymer scaffold surface. This method does not modify scaffold morphology and generates a uniform collagen coating covering fibers throughout the depth of the scaffold. Scaffolds in collagen coated PU (ccPU) groups were covalently coated with bovine type-1 collagen (Nutragen, Advanced Biomatrix, San Diego, Calif.) prior to confocal imaging.

### Mechanical Properties Measurements

Elastic modulus testing was carried out as described in ASTM D3822-07. PLCL scaffolds, Integra, human skin tissue, and human scar tissue samples were cut to 5 cm×5 mm strips and loaded with a 5 mm gap between clamps. Samples were analyzed via microstrain analysis (MSA) (TA Instruments RSA II) at a rate of 0.1 mm/second at room temperature (23° C.) until failure. The initial elastic modulus (within the first 0-200% strain) was analyzed for each sample.

### Confocal Microscopy

Collagen coated PLCL scaffolds were immunostained using anti-collagen-1 antibody and AlexaFluor488 secondary antibody. Stained scaffolds were imaged via confocal microscopy to analyze depth of collagen penetration into the 3D scaffold. Images for quantification of collagen presence were acquired using a Zeiss LSM 510 inverted confocal microscope with 10× magnification.

### Micro-Computed Tomography Analysis

PLCL foams were soaked in Lugol's Iodine (EMS, Hatfield, Pa.) for 3 days and were air dried overnight. Samples were evaluated using a Nikon XTH 225 ST micro-CT scanner. The X-ray source was set to 80 kV and 120 μA. An exposure time of 708 ms was set for each X-ray image. Four X-ray images were then averaged to obtain one 2-D projection. After acquisition, 2-D projections were reconstructed using CT Agent software to provide axial picture cross-sections. After reconstruction, the data was converted into 1400 16-bit picture files with a resolution of <3 μm per pixel. Complete volumes were rendered in Avizo Fire 8.0. Sample porosity was calculated as the ratio of void to solid volume.

### Scanning Electron Microscopy

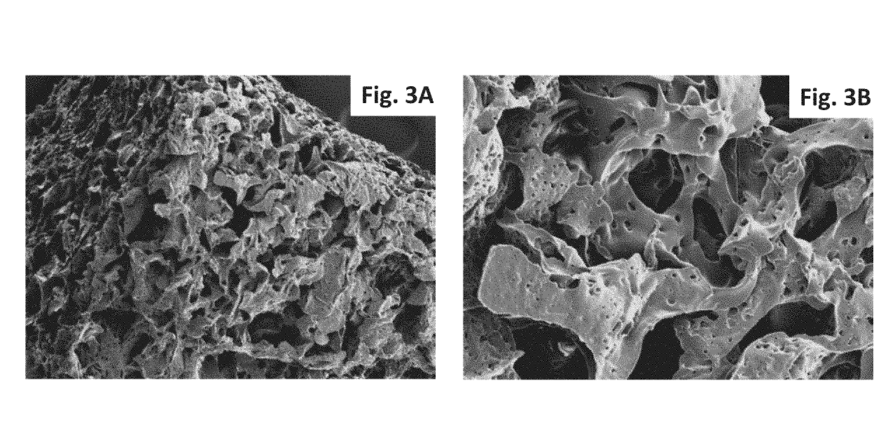

SEM analysis (FIGS. 3A-3B) demonstrated qualitatively uniform porosity throughout the scaffold. The microstructure was seen as an interconnected network of pores among a nanoporous scaffold architecture.

### Mechanical Properties Measurements

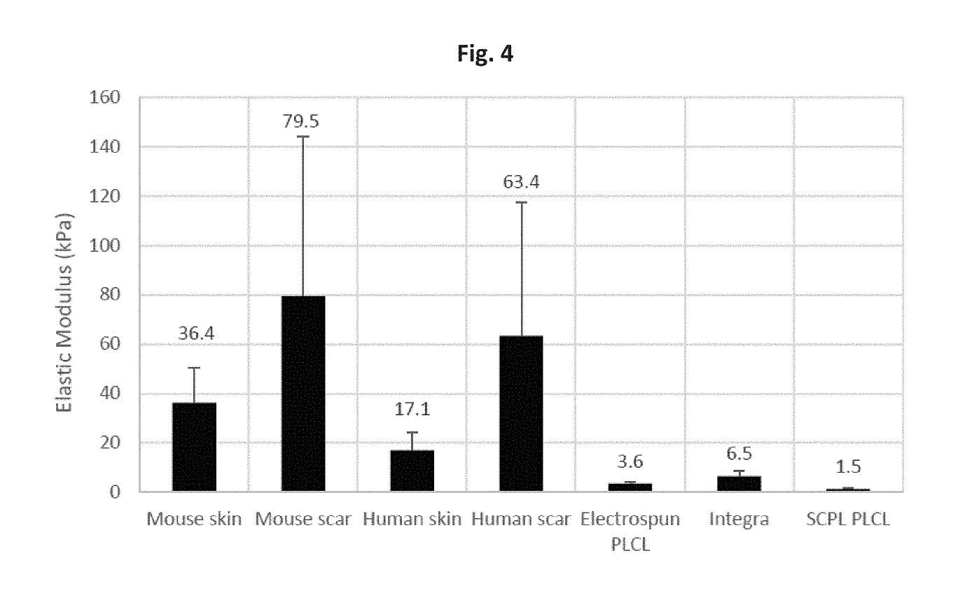

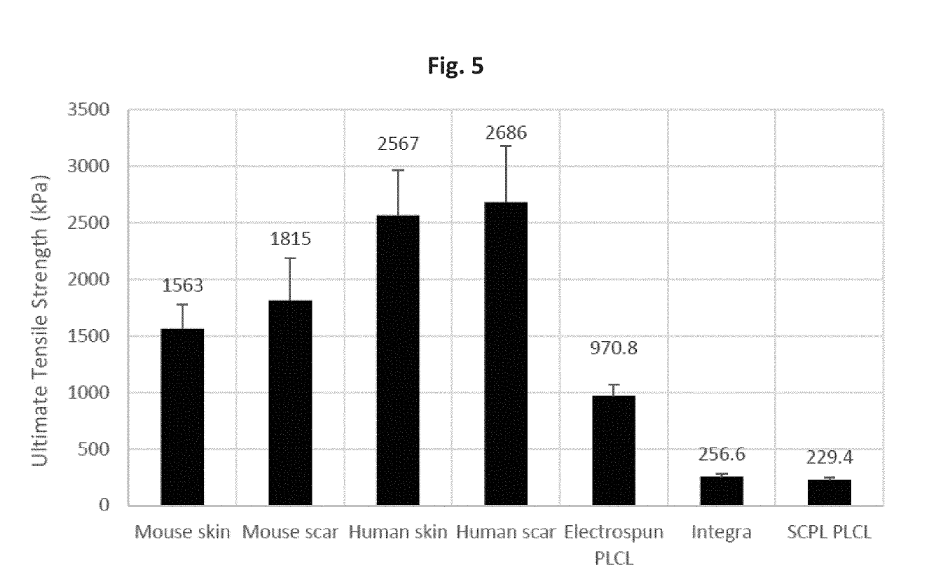

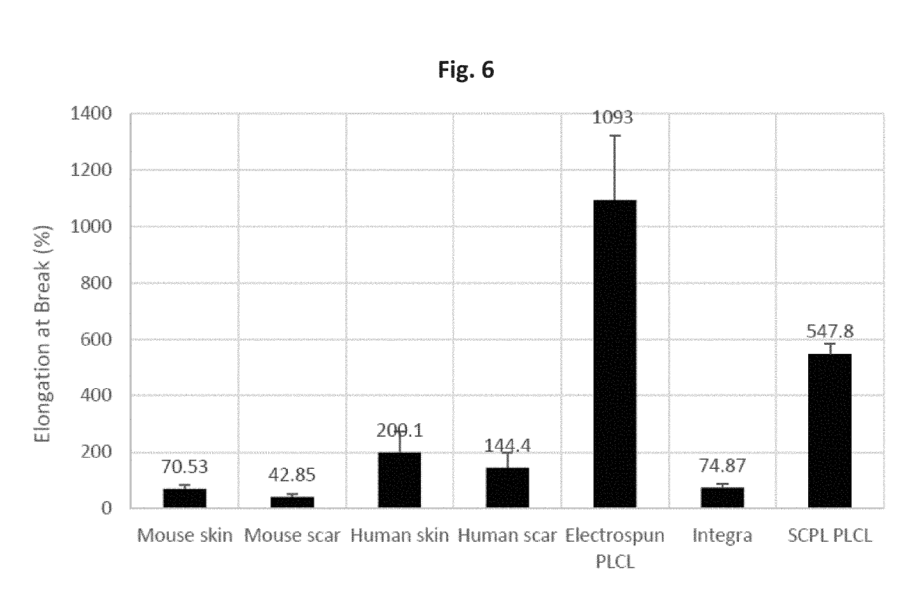

PLCL scaffolds fabricated by solvents casting and particulate leaching (SCPL) exhibited a significantly lower elastic modulus than any other sample tested in the series. The low elastic modulus exemplifies the inherent elasticity of PLCL (FIG. 4), which exhibited a greater elasticity than human skin at the strain rate tested in this study. The ultimate tensile strength was relatively low in comparison to other biomaterials, owing to the porosity of the scaffolds (FIG. 5). Percent elongation at break also confirmed the elasticity of the material, which exhibited an ability to stretch to more than 5-times its original length before breaking (FIG. 6).

### Confocal Microscopy

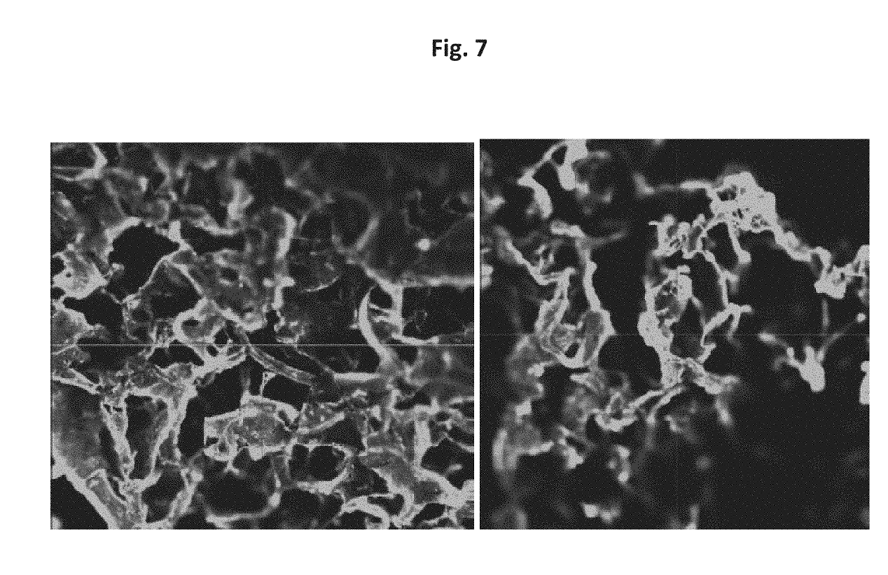

Confocal imaging demonstrated uniform penetration of collagen coating (green) throughout the depth of the scaffold (FIG. 7). Immunofluorescence was highest at the borders of solid material, demonstrating adequate surface bonding of collagen.

### Micro-Computed Tomography

Micro-CT also demonstrated relatively uniform porosity throughout the PLCL scaffolds (FIG. 5). The mean porosity from 4 samples was calculated as 81% (standard deviation 0.5%) by volume averaging.

### Example 2: Comparison of Exemplary Embodiments of the Present Invention

four exemplary porous polymer scaffolds, Samples 1-4 (S1-S4), were prepared. Example 1, described above, provides for the preparation of Sample 1 (51). All samples were prepared from batches of PLCL polymer generously provided by Youngmee Jung, Ph.D. of the Korea Institute of Science and Technology. Samples 1 (51) was prepared from a first size-selection of porogen, i.e., 150-250 microns, and Samples 2-4 (S2-4) were prepared from a second size distribution of sucrose porogen, i.e., 125-250 microns. The weight ratio of sucrose porogen to PLCL polymer was also varied between samples as described in Table 1.

TABLE 1
Comparison of exemplary embodiments of the present invention

| | | | | | | Ultimate | |
|---|---|---|---|---|---|---|---|
| | Su- | Sucrose | | Po- | Elastic | tensile | Elon- |
| | crose | diameters | Polymer | rosity | Modulus | strength | gation |
| | (wt) | (microns) | (wt) | (%) | (kPa) | (kPa) | (%) |
| S1 | 1.48 | 150-250 | 0.5 | 81% | 1.5 | 230 | 548 |
| S2 | 1.79 | 125-250 | 0.15 | 88% | 2.5 | 421 | 495 |
| S3 | 0.80 | 125-250 | 0.15 | 79% | 5.2 | 649 | 485 |
| S4 | 0.46 | 125-200 | 0.15 | 70% | 12.1 | 1013 | 435 |

The Samples prepared demonstrate successful production of PLCL scaffolds ranging from 60-90% porosity, elastic moduli ranging from about 1-13 kPa, ultimate tensile strength of about 200 kPa to 1100 kPa, and percent elongation of about 400-600%. Samples 1-4 (S1-S4) demonstrate that the particular properties may be tailored by those of skill in the art.

Pore interconnectivity is a key to tissue integration and was calculated by dividing the calculated accessible void volume (volume of the voids open to the outside) by the total void volume. Our scaffolds demonstrated at least 99% interconnectivity. Analyses of multiple scaffold slices on the XZ, YZ, and XY axis indicated a 4.7% coefficient of variation in porosity. Scaffolds considered to be non-uniform have been associated with a 48.5% coefficient of variation in pore size. This supports the reproducibility of our manufacturing technique.

### Example 3: Inhibition of Myofibroblast Formation

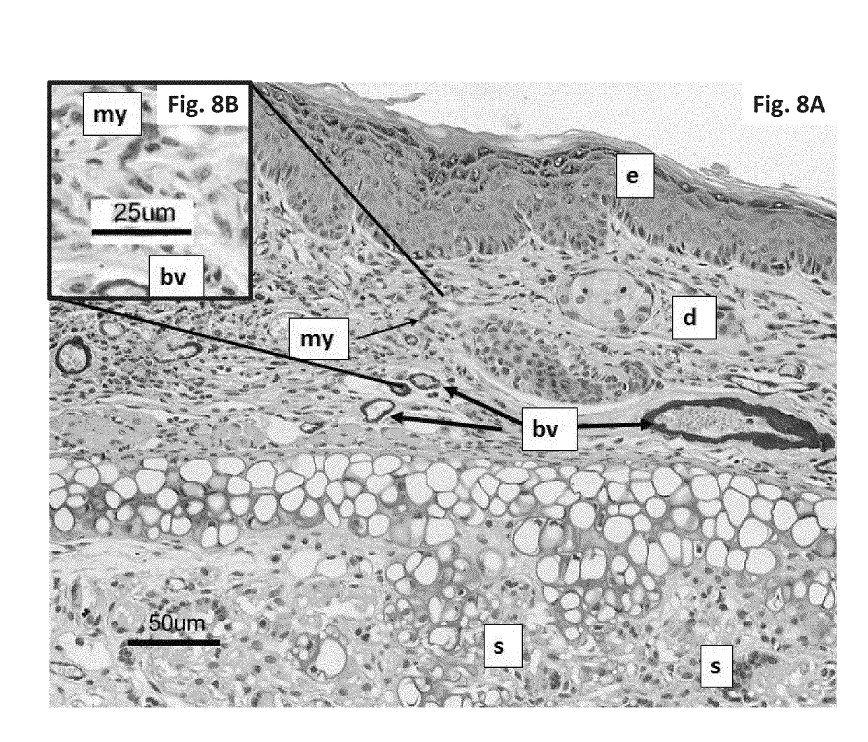

To demonstrate the efficacy of our scaffold at reducing contraction, we implanted PLCL scaffolds in our murine hypertrophic scar model for 2 months. This is the same timeframe at which a collagen based scaffold would disintegrate and adult healing would ensue. Immunostaining for myofibroblasts showed localized αSMA staining peripherally to the scaffold and in blood vessels but noticeably absent within the scaffold (FIGS. 8A-8B). This supports the role of PLCL scaffolds in decreasing mechanical tension.

FIG. 8A shows 20× magnification of hematoxylin stained specimen reveals normal appearing epidermis (e) and dermis (d). The scaffold (s) is seen implanted beneath the skin graft with white spacing indicating scaffold fibers. Myofibroblasts (my) stain positively for αSMA (red) as do blood vessels (bv). FIG. 8B shows 40× magnification demonstrates myofibroblasts as singular red cells without clear association with surrounding vascular structures.

### Example 4: In Vivo Implantation of Wound Healing Products

We created subcutaneous pockets in the flanks of C57BL/6 mice and implant two scaffolds/flank (4 scaffolds/mouse) with one scaffold being cephalad and one being caudal on each flank. There will be 4 mice/time point. Mice will be euthanized at 2 weeks and 4 weeks for a total of 8 mice. We implant PLCL scaffolds with approximately 70, 80, or 90% porosity (Samples 2-4, respectively, as described above) and Integra™ (control scaffolds). Explanted scaffolds will be stained with H&E.

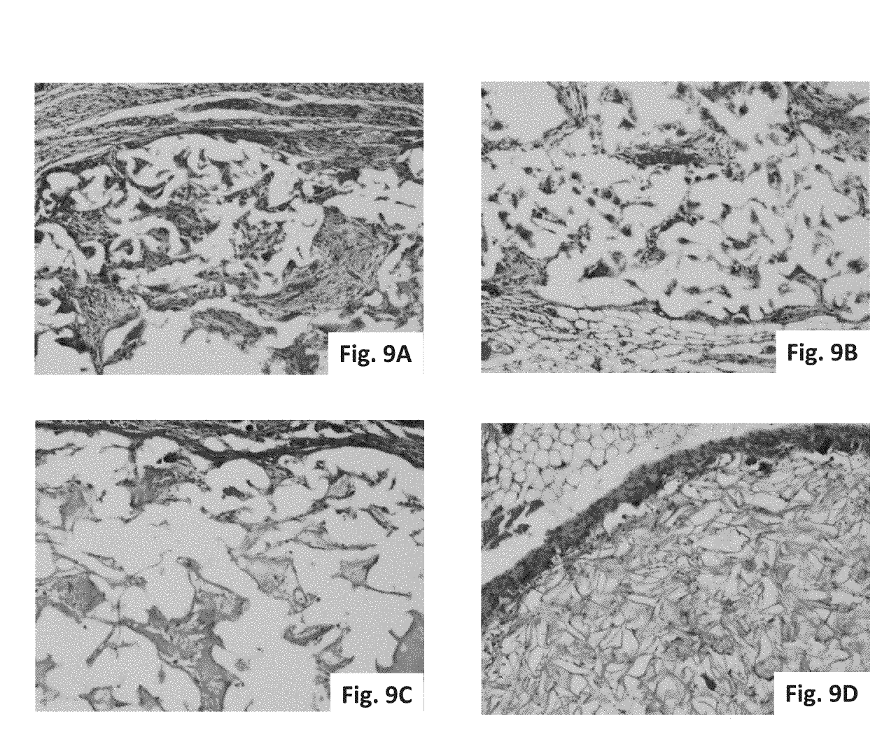

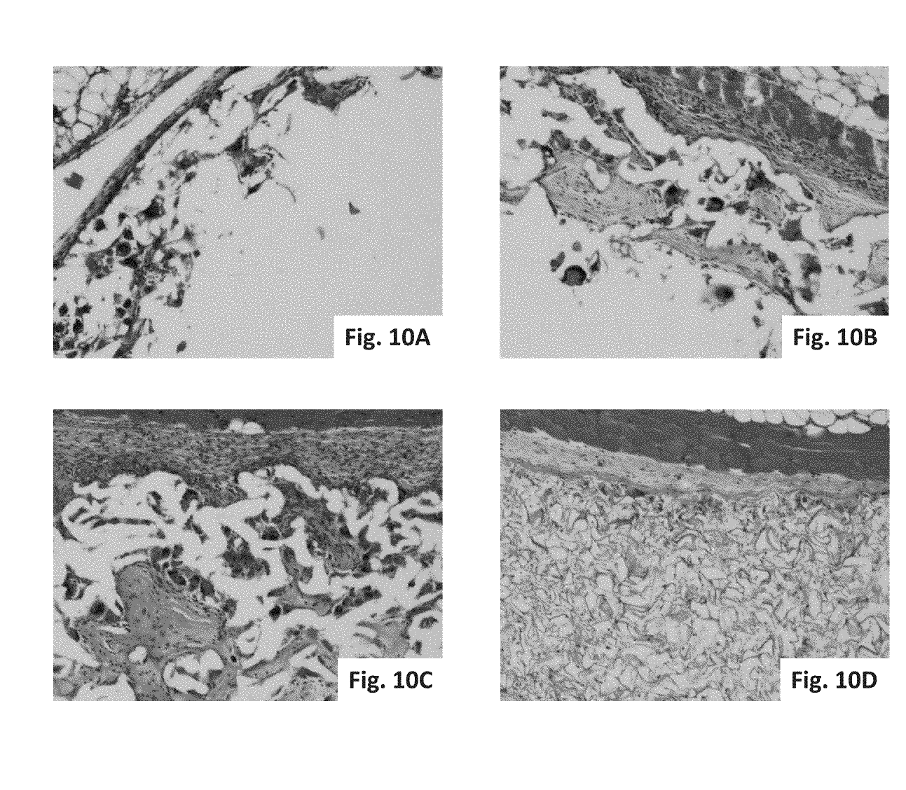

Representative images from a mouse euthanized at 2 weeks (FIGS. 9A-9D) and 4 weeks (FIGS. 10A-10D) are shown. FIG. 9A demonstrates that the 70% porosity PLCL scaffold allows for the presence of mixed inflammatory cells, giant cells, new blood vessels, some fibroblasts, and tissue in-growth inside the implanted scaffold. Outside the implant, there is a very thin capsule and no inflammation. FIG. 9B demonstrates that the 80% porosity PLCL scaffold allows for the presence of mixed inflammatory cells, new blood vessels, some fibroblasts, and more tissue in-growth inside the implanted scaffold than the 70% porosity scaffold. Similarly to FIG. 9A, FIG. 9B shows a very thin capsule and no inflammation. FIG. 9C demonstrates that the 90% porosity PLCL scaffold allows for the presence of mixed inflammatory cells, some fibroblasts, and more tissue in-growth inside the implanted scaffold than the 80% porosity scaffold. Similarly to FIGS. 9A-9B, FIG. 9C shows a very thin capsule and no inflammation. In contrast to FIGS. 9A-9B, FIG. 9D demonstrates that the Integra™ scaffold demonstrates no blood vessels, fibroblasts, or tissue in-growth within the implant. Outside the scaffold, there are mixed inflammatory cells and a bigger capsule than for any of the PLCL implants.

At 4 weeks, the PLCL implants demonstrated greater tissue in-growth and reaction within the implant. FIG. 10A demonstrates that the 70% porosity PLCL scaffold allows for the presence of mixed inflammatory cells, giant cells, new blood vessels, some fibroblasts, and tissue in-growth inside the implanted scaffold. Outside the implant, there is a very thin capsule and no inflammation. FIG. 10B demonstrates that the 80% porosity PLCL scaffold allows for the presence of mixed inflammatory cells, new blood vessels, some fibroblasts, and more tissue in-growth inside the implanted scaffold than the 70% porosity scaffold. Similarly to FIG. 10A, FIG. 10B shows a very thin capsule and no inflammation. FIG. 10C demonstrates that the 90% porosity PLCL scaffold allows for the presence of mixed inflammatory cells, some fibroblasts, and more tissue in-growth inside the implanted scaffold than the 80% porosity scaffold. Similarly to FIGS. 10A-10B, FIG. 10C shows a very thin capsule and no inflammation. In contrast to FIGS. 10A-10B, FIG. 10D demonstrates that the Integra™ scaffold demonstrates absorption of the implant, some macrophage, but no blood vessels, fibroblasts, or tissue in-growth within the implant. Outside the scaffold, no fibrosis is present.

Summarizing all of the mice, fibroblasts, neovascularization, giant cells, neutrophils-focal, and lymphocytes were detected within the PLCL implants and a thin capsule, fibroblasts, and some inflammation was detected outside the PLCL implants at both 2 and 4 weeks. In contrast, the Integra™ implants showed inflammation, thicker capsules, and more fibroblasts outside the implant and minimal reaction within the implant. There was more tissue ingrowth and reaction in 90% scaffolds more than 80% scaffolds more than 70% scaffolds inside the implants where as there were no tissue ingrowth reaction and blood vessels inside the Integra™. The four weeks implants showed more inflammation and tissue reaction than the week two implants in all scaffolds materials with the 90% scaffolds have more tissue ingrowth than the other scaffolds. The Integra™ implants have decreased in size from week two to week four due to absorption.

1. A wound healing product comprising a porous scaffold and collagen bound thereon,
wherein the scaffold comprises a poly(L-lactide-co-ε-caprolactone) polymer (PLCL) substrate having a porosity of between 60% and 95% and a thickness of at least 0.2 mm,
wherein the scaffold has a mean pore diameter of between 50 microns and 250 microns, and
wherein the scaffold has a pore interconnectivity of greater than 80%.

2. (canceled)

3. (canceled)

4. The wound healing product of claim 1, wherein the collagen is collagen I or collagen III.

5. The wound healing product of claim 1, wherein the PLCL substrate comprises a mixture of poly(lactic acid) (PLA) and poly(ε-caprolactone) (PLC) and wherein the PLA and PLC are present in a ratio of 60:40 to 40:60.

6. (canceled)

7. (canceled)

8. (canceled)

9. The wound healing product of claim 1, wherein the scaffold further comprises a porogen.

10. The wound healing product of claim 9, wherein the porogen comprises sucrose.

11. A wound healing product of claim 1 comprising the porous scaffold and collagen I bound thereon,
wherein the scaffold comprises a poly(L-lactide-co-ε-caprolactone) polymer (PLCL) substrate having a porosity of between 80% and 95%, a thickness of at least 1.0 mm, a mean pore diameter of between 50 microns and 250 microns, and a pore interconnectivity of greater than 90%,
wherein the PLCL substrate comprises a mixture of poly(lactic acid) (PLA) and poly(ε-caprolactone) (PLC) and wherein the PLA and PLC are present in a ratio of 60:40 to 40:60,
wherein the scaffold further comprises a porogen.

12. (canceled)

13. (canceled)

14. (canceled)

15. A method for preparing a porous scaffold or a wound healing product, the method comprising:
(a) evaporating a polymer slurry solvent from a polymer slurry within a mold to prepare a polymer substrate surrounding a porogen, wherein the polymer slurry comprises the polymer slurry solvent, the porogen insoluble in the polymer slurry solvent, a solvent-soluble polymer, and a surfactant;
(b) dissolving the porogen with a porogen solvent to prepare a polymer substrate having a plurality of pores;
(c) etching the polymer substrate having a plurality of pores with an oxygen plasma to prepare an etched polymer substrate; and
(d) reacting collagen with the etched polymer substrate to prepare the wound healing product.

16. The method of claim 15, wherein the polymer slurry is prepared by mixing the solvent-soluble polymer and the polymer slurry solvent in a percent weight/weight of the solvent-soluble polymer to the polymer slurry solvent between 10.0% to 20.0%.

17. The method of claim 15, wherein the polymer slurry is prepared by mixing the polymer and porogen in a percent weight/weight of the solvent-soluble polymer to the porogen of 5.0% to 70.0%.

18. (canceled)

19. The method of claim 15, wherein the polymer comprises a poly(L-lactide-co-ε-caprolactone) polymer (PLCL).

20. The method of claim 16, wherein the polymer comprises a poly(L-lactide-co-ε-caprolactone) polymer (PLCL) having a mixture of poly(lactic acid) (PLA) and poly(ε-caprolactone) (PLC) in a ratio of 60:40 to 10:60.

21. The method of claim 15, wherein the porogen comprises a water-soluble sugar or salt.

22. (canceled)

23. (canceled)

24. (canceled)

25. The method of claim 15, wherein the porogen have a mean diameter of between 50 microns to 250 microns.

26. (canceled)

27. (canceled)

28. (canceled)

29. The method of claim 15, wherein the collagen is collagen I or collagen III.

30. The method of claim 15, wherein the reacting step (d) comprises contacting the etched polymer substrate with 1-ethyl-3-(3-dimethylaminopropyl)carbodiimide hydrochloride (EDC) to prepare an O-acylisurea intermediate and displacing the O-acylisurea intermediate with a primary amine of the collagen.

31. The method of claim 30, wherein the reacting step (d) comprises stabilizing an O-acylisurea intermediate with N-hydroxysuccinimide (NHS).

32. The method of claim 15,
wherein the reacting step (d) comprises:
(i) contacting the etched polymer substrate with 1-Ethyl-3-(3-dimethylaminopropyl)carbodiimide hydrochloride (EDC) to prepare an O-acylisurea intermediate;
(ii) stabilizing an O-acylisurea intermediate with N-hydroxysuccinimide (NHS); and
(iii) displacing the O-acylisurea intermediate with a primary amine of collagen I to prepare the wound healing product,
wherein the polymer is a poly(L-lactide-co-ε-caprolactone) polymer (PLCL) having a mixture of poly(lactic acid) (PLA) and poly(ε-caprolactone) (PLC) in a ratio of 60:40 to 10:60, and
wherein the porogen comprises sucrose granules selected by size-exclusion to have a mean diameter of 50 microns to 250 microns.

33. The method of claim 15, wherein the etching step comprises contacting the porous scaffold with an oxygen plasma having an sufficient power for a sufficient time to oxidize the surface of the porous scaffold.

34. (canceled)

35. (canceled)

36. (canceled)

37. (canceled)

38. (canceled)

39. (canceled)

40. (canceled)

41. A method of promoting wound healing in a subject comprising providing the wound healing product of claim 1, wherein the wound healing product is configured to be implanted at the site of a wound or beneath an applied skin graft of the subject.

42. (canceled)

43. The method of claim 41, wherein the wound is a chronic wound, a surgical wound, or fibrosis.

44. (canceled)

45. (canceled)

46. (canceled)
